(19) United States
(12) Patent Application Publication
(10) Pub. No.: US 2026/0248918 A1
(43) Pub. Date: Aug. 27, 2026

(54) COMPOSITIONS AND METHODS OF SUPPRESSING AVERSIVENESS OF PHARMACEUTICALS AND INGESTIBLE MATERIALS

(71) Applicant: Monell Chemical Senses Center, Philadelphia, PA (US)

(72) Inventors: Erik M. Schwiebert, Birmingham, AL (US); John H. Streiff, Birmingham, AL (US); Peihua Jiang, Clarksville, MD (US); Paul A.S. Breslin, Pittsburgh, PA (US)

(21) Appl. No.: 19/649,954

(22) Filed: Apr. 16, 2026

Related U.S. Application Data
(63) Continuation of application No. 17/641,853, filed on Sep. 9, 2020, now abandoned.
(60) Provisional application No. 62/898,686, filed on Sep. 11, 2019.

(51) Int. Cl. A61K 47/22; A61K 45/06

(52) U.S. Cl. CPC A61K 47/22; A61K 45/06

(57) ABSTRACT

Provided herein are compositions and methods of ameliorating the pharmaceutical aversiveness of active pharmaceutical ingredient (API) by administering the API after or substantially simultaneously with a flavone compound.

### CROSS-REFERENCE TO RELATED APPLICATIONS

This application is a continuation of U.S. patent application Ser. No. 17/641,853, filed Mar. 10, 2022, which is a national stage entry under 35 U.S.C. § 371 of International Patent Application No. PCT/US2020/049797, filed Sep. 9, 2020, which claims the benefit under 35 U.S.C. § 119(e) of U.S. Provisional Patent Application No. 62/898,686, filed Sep. 11, 2019. The foregoing applications are incorporated by reference herein in their entireties.

### BACKGROUND OF THE INVENTION

Significant numbers of drugs and active pharmaceutical ingredients (APIs) on the market and in development may cause aversiveness upon oral administration, e.g., bitter taste, mouth/throat irritation, and nausea, not only to children but also to many adults. Such drugs are necessary to treat global diseases such as infections with bacteria, plasmodia, and helminths, many of which are fatal. Thus, acceptable palatability of oral medicinal products is of great importance to facilitate patient adherence with a drug regimen, particularly in underdeveloped countries where medical supervision of such adherence is lacking. Unlike adults who can swallow taste-masked tablets, children are exposed to other formulations (e.g., liquid). When in liquid form, drugs can be highly aversive and children, particularly have demonstrated little tolerance of these formulations resulting in problems with compliance. If drug compliance is not improved, millions of children will continue to die from drug-treatable diseases. This has been recognized by regulatory authorities and is becoming a key aspect of pediatric pharmaceutical development studies. See, e.g., Walsh et al, “Playing hide and seek with poorly tasting pediatric medicines: Do not forget the excipients.” Advanced Drug Delivery Reviews. 73 (2014) 14-33; and Mennella et al., “Optimizing Oral Medications for Children.” Clin Ther. 2008 Nov. 30(11): 2120-2132.

Several approaches have been utilized to mask unpleasant tastes of APIs in pediatric oral dosage forms. However, few successes have been achieved mainly due to high complexity of taste receptors and signal pathways involved in taste sensations as well as little understanding thereof. While sweeteners and flavors are the intuitive choice, such additives, when found to be effective, harm dental health or cannot be provided to diabetic children. Similarly, when compounds have been previously employed to ameliorate bitterness, the degree of bitterness suppression, if any, varied widely across bitter substances. See, e.g., P. A. S. Breslin and G. K. Beauchamp, “Suppression of Bitterness by Sodium: Variation Among Bitter Taste Stimuli.” Chemical Senses, Volume 20, Issue 6, 1 Dec. 1995, Pages 609-623, doi.org/10.1093/chemse/20.6.609. Whether or not the use of any compound can suppress bitterness of any composition does not appear to be predictable from known publications. See, for example, the disclosures of both U.S. Pat. No. 5,580,545 Washino et al. which discloses certain flavones as taste modifiers and Roland, W S U et al. PLOS One, April 2014, 9 (4): 394451 among others.

Compositions and methods to effectively ameliorate pharmaceutical aversiveness continue to be needed.

### SUMMARY OF THE INVENTION

In one aspect, a composition comprises a compound of Formula A below:

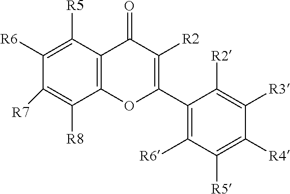

wherein R2, R5, R6, R7, R8, R2′, R3′, R4′, R5′, and R6′, are independently selected from H, OH, a C₁ to C₁₀ alkyl, C₂ to C₁₀ alkenyl, C₂ to C₁₀ alkynyl; C₁ to C₁₀ alkoxy, or a substituted alkyl, alkenyl or alkynyl, or a halogen, and a carrier or excipient, which is pharmaceutically acceptable or safe for human consumption, wherein the composition suppresses the aversive taste of an ingestible material.

In another aspect, a composition is provided comprising a compound of Formula A, as described above and a suitable pharmaceutically acceptable carrier or excipient. The composition suppresses the taste of an active pharmaceutical ingredient (API) which causes aversiveness in a subject upon oral administration. In one embodiment, the aversiveness is selected from the group consisting of bitterness, sourness, astringency and nausea. In one embodiment, the compound of Formula A is a flavone. In another aspect, the compound is 6-methylflavone. In another aspect, the composition contains a compound of Formula A and another blocker of the same or a different taste receptor or a cocktail of different taste receptor blockers. In still another embodiment, the composition comprises a compound of Formula A, and optionally contains the API.

In another aspect, a composition comprises tenofovir alafenamide fumarate (TAF) and a compound of Formula A. In one embodiment, the composition comprises TAF and 6-methylflavone.

In still another aspect, a composition comprising a flavone, i.e., 6-methylflavone, in a carrier or excipient in a pre-rinse for treatment of a subject prior to contact with said API.

In yet another aspect, a liquid pre-rinse composition comprises a compound capable of blocking the activity of TAS2R taste receptors or other taste receptors that mediate an aversive response from a mammalian subject upon contact with the oral cavity, and a suitable pharmaceutically acceptable carrier or excipient, wherein the composition suppresses the aversive taste of an ingestible composition or compound in a suitable ingestible carrier and with optional excipient components comprising one or more of sodium salts, zinc salts, sophorolipids, components derived from milk, milk, and water.

In another aspect, the pre-rinse composition is administered to the subject before administration of the API, substantially simultaneous with the API or in admixture with the API.

In another embodiment a liquid pre-rinse composition comprises:
(a) one or more compounds of Formula A as described herein;
(b) carrier and excipient components comprising one or more of sodium salts, zinc salts, sophorolipids, components derived from milk, milk, and water; and
(c) optional TASR2 blockers other than (a). In another embodiment of this pre-rinse composition, it contains an API.

In still another aspect, a kit containing the components of the pre-rinse, including instructions for a drug regimen involving the kit components is provided.

In another aspect, a method of suppressing the taste of an active pharmaceutical ingredient (API) which causes aversiveness in a subject upon oral administration comprises administering to a mammalian subject in need thereof a pre-rinse composition comprising: one or more compounds of Formula A as described herein; optional carrier and excipient components comprising one or more of sodium salts, zinc salts, sophorolipids, components derived from milk, milk, and water; one or more optional TAS2R blockers other than that of Formula A; and an optional API.

In another aspect, a method of suppressing the taste of an active pharmaceutical ingredient (API) which causes aversiveness in a subject upon oral administration comprises administering a composition as described herein to said subject. In various embodiments, the composition may be a liquid, gel, semi-solid, solid or lozenge. In one embodiment, composition is administered before or substantially at the same time as the API. In another aspects, the composition contains the API and so is administered simultaneously in admixture therewith.

In one aspect, a method is provided herein to improve drug regimen compliance and/or suppress aversiveness of an API orally administrated to a subject. In one embodiment, the method comprises administering the API and an effective amount of compound of Formula A orally in the subject.

In still another aspect, methods are provided for reducing the aversive qualities of a drug by formulating the API of the drug with an effective amount of a compound of Formula A which suppresses the aversiveness.

In yet a further aspect, a method of suppressing the taste of an ingestible material which causes aversiveness in a mammalian subject upon oral administration comprises administering to a mammalian subject in need thereof a composition comprising one or more compounds of Formula A as described herein and a suitable carrier and excipients safe for human ingestion.

Other aspects and embodiments will be readily apparent based on the information described herein.

### BRIEF DESCRIPTION OF THE DRAWINGS

FIG. 1 is a schematic of the labeled magnitude scale (LMS) which is used to measure the strength of sensations. It is a ratio scale (i.e., 40 is twice as intense as 20). Even weak levels of negative sensations such as bitterness are too strong to be acceptable for young children.

FIG. 2 shows four graphs of the sensory properties e.g., bitterness, sourness astringency and nausea sensation, measured as perceived intensity (gLMS) of ascending concentrations (mg/mL) of Piperaquine. The number of subjects reporting was 14.

FIG. 3 shows five graphs of the sensory properties, e.g., bitterness, sourness astringency, nausea sensation and burning sensation, measured as perceived intensity (LMS) of ascending concentrations (mg/mL) of tenofovir alafenamide fumarate (TAF). The number of subjects reporting was 8.

FIG. 4 is a table showing response profiles of TAS2Rs to indicated target compounds at indicated concentrations. Praziquantel: 0.75 mM, L-praziquantel: 0.1 mM, Zinc sulfate: 20 mM, Dihydroartemisinin: 2 mM (not fully dissolved), Piperaquine, 0.5 mM, Tenofovir (TAF), 1 mM and Ritonavir, 1 mM. Symbols: + means that there was activation; a − negative sign means that there was no significant activation of the receptor; a “larger + sign” means that receptor(s) were most sensitive to the compound (activated at lower concentrations.

FIG. 5A is a bar graph showing that the blocker 6-methylflavone (indicated by the arrow number 2) blocks the activity of taste receptor TAS2R39 to Piperaquine. Other blockers are unidentified compounds identified by the numbers following the concentrations under the X axis. Five candidates from the HTS screen blocked the activity of TAS2R39 to Piperaquine. 6-methylflavone (#2) shows strong inhibition of TAS2R39. It did not inhibit TAS2R1 (data not shown).

FIG. 5B is a graph plotting the activity of taste receptor HEK/TAS2R39 in the presence of 0.5 mM Piperaquine (bold black line); the combination of 0.5 mM Piperaquine and 0.1 mM of 6-methylflavone (light gray line), and a control with 0.1 mM of the blocker 6-methylflavone (dotted line).

FIG. 6 is a graph showing that piperaquine (8 mg/mL) treated with 100 μM 6-methylflavone (Compound 2) affected average perceived bitterness intensity. The numbers of subjects treated is represented by the number n above each bar.

FIG. 7A is a bar graph showing that the blocker 6-methylflavone (indicated by the arrow number 2) completely inhibited the activity of taste receptor TAS2R39 in response to Tenofovir (1 mM). Other blockers are identified by the numbers following the concentrations under the X axis.

FIG. 7B is a graph plotting the activity of taste receptor TAS2R39 in the presence of 1 mM Tenofovir (bold black line); the combination of 1 mM Tenovir and 0.1 mM of 6-methylflavone (light gray line), and a control with 0.1 mM of the blocker 6-methylflavone (dotted line). 6 methylflavone did not inhibit the response of TAS2R1 to TAF (data not shown).

FIGS. 8A and 8B are bar graphs showing that one subject showed significant suppression of TAF bitterness in the presence of 6-methylflavone (0.5 mg/mL TAF treated with and without 100 μM 6-methylflavone (#2)). Some individuals show significant suppression of TAF bitterness in the presence of 6-methylflavone.

FIGS. 9A and 9B are bar graphs showing that one subject did not show significant suppression of TAF bitterness when response to TAF alone vs. TAF with a pre-rinse vs. TAF with a pre-rinse containing 6-methylflavone (0.5 mg/mL TAF treated with and without 100 μM 6-methylflavone (#2)).

FIGS. 10A and 10B are bar graphs showing two repetitions of a bitterness response of one subject first treated with 0.5 mg/mL TAF alone, then treated with TAF with a pre-rinse containing 100 μM or 1 mM 6-Methylflavone (the blocker), followed by a second administration of an admixture of TAF and 6-Methylflavone. This subject showed no bitterness suppression at the lower concentration (i.e., 100 μM) of the blocker.

FIGS. 11A and 11B are bar graphs showing two repetitions of a bitterness response of another subject first treated with 0.5 mg/mL TAF alone, then treated with TAF with a pre-rinse containing 100 μM or 1 mM 6-Methylflavone, followed by administration of an admixture of TAF and 100 μM or mM 6-Methylflavone. The 6-Methylflavone blocker worked at the lower concentration (100 μM) for this subject and worked even better at the higher concentration (1 mM).

FIGS. 12A and 12B show concentration-response curves or relationships (CRCs) for antagonism of TAF or piperaquine stimulation of hTBEC cells with an optimal concentration of each bitter-tasting medicine in vitro. Each inhibition curve was fitted to the data so as to calculate an IC50 or the concentration that inhibits 50% of the signal. A Hill slope was calculated, along with the % maximal inhibition and the concentration of antagonist, 6-methylflavone, (indicated by the ref ST069348) required to fully block the response in the assay. These metrics were slightly different for antagonism by 6 -methylflavone of TAF versus piperaquine. FIG. 12A is a graph showing the suppression of human taste bud cells (hTBEC) cells with 6-methylflavone added to 300 micromolar TAF stimulated cells. IC50 is 7.7 micromolar for 6-methylflavone antagonism of TAF stimulation. Hill slope is 2.18; % maximum inhibition of >95% at 50 μM. FIG. 12B is a graph showing the suppression of hTBEC cells with 6-methylflavone added to 100 micromolar Piperaquine stimulated cells. IC50 is 2.0 μM for 6-methylflavone antagonism of Piperaquine stimulation. Hill slope is 1.992; % maximum inhibition of >95% at 50 μM.

### DETAILED DESCRIPTION OF THE INVENTION

Provided herein are compositions and methods of ameliorating pharmaceutical aversiveness thus facilitating patient adherence to a drug regimen. The aversiveness is ameliorated when certain compounds are added to or administered in a regimen with selected unpleasant-tasting pharmaceutical compounds (e.g., drugs or APIs, as discussed and defined below). In other aspects, the compositions described herein can also be used to suppress or reduce an aversive response to other ingestible materials, including foods, beverages, vitamins, over-the counter products such as oral care products, and the like.

Using human taste bud cells in a small-scale screening study, the inventors successfully identified a class of compounds, flavones, and particularly 6-methylflavone, that not only inhibits API activity in human taste cells and in a human bitter taste receptor (TAS2R39) but also reduces perceived bitterness in human sensory testing. As disclosed below in more detail, in one embodiment a flavone compound was identified that inhibits Piperaquine and TAF activity in human taste bud cells (hTBEC) and in a human bitter receptor (TAS2R39). The same compound also significantly reduced the drugs' perceived bitterness. Blockers for specific bitter taste receptors are used in combination with other taste modifiers (e.g. Na and Zn salts) and sweeteners that can render many APIs palatable. In another aspect, a combination of inhibitors of TAS2R1 and TAS2R39 is contemplated.

A variety of compositions and methods for reducing or suppressing aversive reactions to pharmaceuticals and thus encouraging drug compliance, and combinations of the blocker compounds of Formula A and API may be selected and combined in effective amounts. Similarly, various combinations of the compounds of Formula A with other TAS2R blocker compounds can be used to reduce or suppress aversive reactions to other ingestible materials as provided herein

### 1. Components of the Methods and Compositions

Unless defined otherwise in this specification, technical and scientific terms used herein have the same meaning as commonly understood by one of ordinary skill in the art and by reference to published texts, which provide one skilled in the art with a general guide to many of the terms used in the present application.

The terms “amelioration”, “reduction”, “decrease”, “suppression”, or any grammatical variation thereof as used herein may refer to at least about 5%, at least about 10%, at least about 20%, at least about 30%, at least about 40%, at least about 50%, at least about 60%, at least about 70%, at least about 80%, at least about 90%, or at least about 95% of subjects tested showing an intensity of indicated sensation lower than the reference given. In certain embodiment, the terms “amelioration”, “reduction”, “decrease”, “suppression”, or any grammatical variation thereof as used herein may refer to an intensity of indicated sensation which is less than about 95%, less than about 90%, less than about 80%, less than about 70%, less than about 60%, less than about 50%, less than about 40%, less than about 30%, less than about 20%, less than about 10%, or less than about 5% of the reference given.

Flavones are aromatic, heterocyclic compounds commonly found in many higher plants and are generally characterized as having a closed three-carbon bridge (i.e. a third ring structure) wherein the second or B-ring is attached at the carbon adjacent the ketone (i.e. the C-2 carbon) and as further having a double bond between the C-2 and C-3 carbons. By way of non-limiting example, flavones believed suitable for use in the present invention include, but are not limited to, compounds have the structure of Formula A

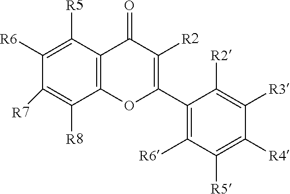

wherein R2, R5, R6, R7, R8, R2′, R3′, R4′, R5′, and R6′ are independently selected from H, OH, a C1 to C10 alkyl, C2 to C10 alkenyl, C2 to C10 alkynyl; C1 to C10 alkoxy, or a substituted alkyl, alkenyl or alkynyl, or a halogen, and a suitable pharmaceutically acceptable carrier or excipient. The composition suppresses the bitter taste of an active pharmaceutical ingredient (API) which causes aversiveness in a subject upon oral administration.

In one embodiment, the compound of Formula A has the following substituents: R6 is methyl and R2, R5, R7, R8, R2′, R3′, R4′, R5′, and R6′ are each hydrogen and is 6-methylflavone. 6-methylflavone is active in bioassays for in vitro affinity against Benzodiazepine receptor binding to rat cortical membranes (gamma-aminobutyric acid receptor subunit); has the ability to inhibit [3H]-Ro-15-1788 binding to Gamma-aminobutyric acid A receptor alpha in vitro; has agonist activity at androgen receptor in human MDA-kb2 cells assessed as stimulation of luciferase activity by luciferase reporter gene assay; and has antagonist activity at glucocorticoid receptor in human MDA-kb2 cells (see e.g., PubChem SID 103369714/CID 689013). However, it was specifically reported in Roland WSU et al, PLOS One, (2014) 6-Methoxyflavanones as Bitter Taste Receptor Blockers for hTAS2R39. PLOS ONE 9(4): e94451 doi: 10.1371/journal.pone. 0094451 that “the compound 6-methoxyflavone was unable to inhibit hTAS2R39 activation (data not shown), which indicated that absence of a double bond in the C-ring is essential for inhibition.”

In another embodiment, the compound of Formula A has the following substituents: R5 is OH, R3′ is OH, R4′ is OH and R2, R7, R8, R2′, R5′, and R6′ are each hydrogen. In yet another embodiment, the compound of Formula A has the following substituents: R5 is OCH₃, and R2, R7, R8, R2′, R3′, R4′, R5′, and R6′ are each hydrogen. In yet another embodiment, the compound of Formula A has the following substituents: R6 is OCH₃, R4′ is OH, and R2, R5, R7, R8, R2′, R3′, R5′, and R6′ are each hydrogen. In still another embodiment, the compound of Formula A has the following substituents: R5 is OCH₃, R7 is OCH₃ and R4′ is OCH₃ and R2, R8, R2′, R3′, R5′, and R6′ are each hydrogen or wherein R4′ and R5 are each OCH₃ and the remaining R groups are hydrogen. These compounds are also named 5′3′4′ trihydroxyflavone, 5 methoxyflavone, 4′-hydroxyl-6-methoxyflavone, 5, 7, 4′ trimethoxyflavone or 4′, 5 dimethoxyflavone. Still other known flavones falling within Formula A are anticipated to perform as an aversive taste blocker in the manner of 6-methylflavone.

A suitable pharmaceutically acceptable carrier or excipient, or carriers and excipients that are safe for human ingestion may include without limitation, a diluent, an excipient, a vector, a stabilizer, a buffer, a preservative, a sweetener, a flavor, a taste receptor antagonist, a taste transduction cascade blocker, and/or an adjuvant. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. Still other carriers include those identified in texts such as Remington's Pharmaceutical Sciences, 17^{th} edit., 1985 Gennaro, AR eds., Mack Publishing Co, Easton PA; and Handbook of Pharmaceutical Excipients, 6^{th} edit., 2009 Rowe R C et al, eds, Pharmaceutical Press, incorporated by reference herein.

In certain embodiments, the carrier is water. In other embodiments the carrier is milk or a milk product. In certain embodiments, the carrier or excipient includes salts, such as zinc salts or sodium salts, including without limitation, sodium chloride, zinc chloride, sodium gluconate, zinc gluconate, sodium glutamate, zinc glutamate, sodium adenosine monophosphate, zinc adenosine monophosphate, sodium hydroxide and zinc hydroxide).

In still other embodiments the carrier includes sophorolipids (SL), i.e., a surface-active glycolipid compound of microbial origin which consists of a hydrophilic carbohydrate head, sophorose (2-O-β-D-glucopyranosyl-β-D glucopyranose) attached through a glycosidic linkage to a hydrophobic fatty acid tail of 14 to 24 carbon atoms. The hydrophilic portion, sophorose, is a glucose di-saccharide with an unusual β-1,2 bond and can be acetylated on the 6′- and/or 6″-positions. The specific location of those chemical bonds is dependent on the microbial strain used to produce the SLs, including but not limited to strains of the yeast Candida bombicola. The hydrophobic tail, i.e., the lipid component, is in the embodiment exemplified below an 18-carbon chain fatty acid, having one unsaturated bond, produced using oleic acid. Other fatty acids may be used in the production of sophorolipids useful herein and may have one or two saturated bonds. In still other embodiments, the structural conformation of sophorolipid produced by C. bombicola from glucose and palmitic acid, oleic acid or stearic acid is as a lactone where the carboxylic acid group of the fatty acid is esterified to the disaccharide ring at carbon 4. See the discussion and references cited in US Patent Application Publication US2017/0189533, incorporated by reference herein. In one embodiment, the sophorolipid is produced using the fatty acid, oleic acid. In another embodiment, the sophorolipid is produced using the fatty acid, palmitic acid. In yet another embodiment, the sophorolipid is produced using the fatty acid, stearic acid. In the Example below, the sophorolipid used has an oleic acid tail. Alternatively, suitable sophorolipids may be prepared as described herein or purchased or obtained from publicly available sources. SL are produced by microorganisms (Candida batistae CBS 8550, Rhodotorula bogoriensis, Candida floricola TM1502, Candida riodocensis, Candida rugosa, Candida kuoi, Candida stellate, Candida tropicalis, Cryptococcus sp. VITGBN2, Cyberlindnera samutprakarnensis JP52, Pichia anomala PY1, Rhodotorula muciliginosa, Candida bombicola, Candida apicola, Torulopsis gropengiesseri, Torulopsis petrophilum and Wickerhamiella domercqiae Y2A.

As used herein, the term “aversiveness” or any grammatical variation thereof refers to an unpleasant sensation selected from the group consisting of bitter taste (bitterness), sourness, astringency, nausea and burning or stinging sensation. As used herein, the term “sensation” refers to a perception associated with stimulation of a drug in gastrointestinal tract (e.g., mouth or tongue), including without limitation, taste, astringency and nausea. Conventional methods of quantifying an aversiveness are known to one of skill in the art. See, e.g., Breslin et al, “Suppression of Bitterness by Sodium: Variation Among Bitter Taste Stimuli.” Chemical Senses, Volume 20, Issue 6, 1 Dec. 1995, Pages 609-623, doi.org/10.1093/chemse/20.6.609; Peyrot des Gachons, C. et al, (2011). “Bitter taste induces nausea.” Current Biology, 21, R247-248 PMID 21481757; Keast, R. S. J. and P. A. S. Breslin. (2005) “Bitterness suppression with zinc sulfate and Na-cyclamate: a model of combined peripheral and central neural approaches to flavor modification”. Pharmaceutical Science, 22, 1970-1977. PMID: 16132352; Breslin P A, and Tharp C D. “Reduction of saltiness and bitterness after a chlorhexidine rinse”, Chem Senses. 2001 February; 26(2):105-16; and Keast, R. S. J. and P. A. S. Breslin. (2002) “Modifying the bitterness of selected oral pharmaceuticals with cation and anion series of compounds.” Pharmaceutical Research, 19, 1019-1026. PMID: 12180534, each of which is incorporated herein in its entirety. Methods of quantifying an aversiveness are also provided herein in the Examples.

By “inhibiting or reducing an aversive taste or sensation” is meant that the method or composition described herein can decrease the sensation in the oral cavity when compared to the sensation achieved by the drug or API or other ingestible material which is not associated with a compound of Formula A. In one embodiment, the method described herein and/or the use of a composition to which a compound of Formula A is added as described herein, reduces the aversive sensation of the composition by up to 25% compared to the original composition without a compound of Formula A. In another embodiment, the method or composition inhibits the aversive sensation by up to 50%. In still other embodiments, the methods or compositions described herein reduce or inhibit the aversive sensation by at least 5%, 10%, 15%, 20%, 25%, 30%, 35%, 40%, 45%, 50%, 55%, 60%, 65%, 70%, 75%, 80%, 85%, 90%, 95% or up to 100% including whole and fractional percentages between each number indicated herein.

As used herein, the term “administration” or any grammatical variations thereof refers to delivery of composition described herein to a subject. “Oral administration” or any grammatical variations thereof refers to an administration where composition described herein is taken by the subject through mouth. In one embodiment, oral administration may include without limitation, enteral administration, wherein the composition is taken through mouth and absorbed in the gastrointestinal tract; buccal administration wherein the composition is dissolved inside the cheek; sublabial administration, wherein the composition is dissolved under the lip; and sublingual administration, wherein the composition is dissolved under the tongue. Furthermore, the form of administration can be liquid (i.e., solutions or suspensions), soluble or dispersible tablets, oral wafers, chewable tablets, or orodispersible tablets.

As used herein, “an effective amount” refers to a concentration of a compound in the formulation of a drug, a concentration of a compound in mouth, or a molar ratio of a drug (API) to compound of Formula A, which suppresses aversiveness of the orally administrated drug compared to the oral administration of the drug only. In one embodiment, “an effective amount” might also refer to an amount of a compound when mixed with a drug and administrated orally, suppresses aversiveness of the drug. In a further embodiment, the compound is mixed with a drug in a liquid formulation. In one embodiment, the effective amount of the compound of Formula A is about 0.1 μM to about 100 μM (0.1 mM), about 0.1 mM to about 1 mM, and optionally in excess of 10 to 100 mM. In another embodiment, the effective amount is about 100 mM. In certain embodiment, the effective amount is about 0.1 mg to about 1 mg, about 1 mg to about 5 mg, about 5 mg to about 10 mg, about 10 mg to about 50 mg, about 50 mg to about 100 mg, about 100 mg to about 250 mg, about 250 mg to about 500 mg, about 500 mg to about 1 g, about 1 g to about 2 g, about 2 g to about 5 g, about 5 g to about 10 g.

In certain embodiments, the effective amount is defined as the molar ratio of API (or Drug) to compound of Formula A. In one embodiment, the molar ratio is about 0.00003 (3×10⁻⁵) to about 0.5 (5×10⁻¹), or any number including and between these numbers. In one embodiment, the effective amount is a molar ratio of the API/drug to the compound of about 0.0001 (1×10⁻⁴) to about 0.0010 (1×10⁻³) or any number including and between these numbers. In another embodiment, the effective amount is a molar ratio of the API/drug to the compound of about 0.001 (1×10⁻³) to about 0.010 (1×10⁻²) or any number including and between these numbers. In yet another embodiment, the effective amount is a molar ratio of the API/drug to the compound of about 0.01 to about 0.10 or any number including and between these numbers. In a further embodiment, the effective amount is a molar ratio of the API/drug to the compound of about 0.1 to about 0.5 or any number including and between these numbers. Still other dosages may be useful in the compositions and methods described herein.

By “physiological or behavioral assay for an aversive sensation” is meant an assay relying on physiological or behavioral reactions in response to increased or decreased aversive sensation of a compound applied to the oral cavity of a mammalian test animal or human. Examples of known assays include primarily sip and spit taste assays. See, e.g., Delwiche et al, Food Quality and Preference, 1996 March; 7(3-4)293-7. Other suitable assays are known to those of skill in the art and may be applied in the methods described below.

By “ingestible material” is meant any product which is placed in the oral cavity for purposes of medical or dental treatment or for purposes of consumption. Such material includes without limitation, any pharmaceutical drug for oral systemic use, foodstuffs, beverages, sore throat or cough lozenges, vitamins, oral care or dental care products, such as toothpaste and mouthwash, topical anesthetics and the like.

By “ingestible vehicles” is meant ingestible or partly ingestible vehicles such as confectionery bulking agents which include hard and soft vehicles such as, for example, tablets, suspensions, chewable candies or gums, lozenges and so forth. Exemplary ingestible vehicles and compositions for use therein are described in Remington: The Science and Practice of Pharmacy, 20th Edition (Lippincott Williams & Wilkins 2001); Goodman & Gilman's The Pharmaceutical Basis of Therapeutics, 10th Edition (McGraw-Hill Professional, 2001) and The United States Pharmacopoeia: The National Formulary (US Pharmacopoeia Convention, Inc.).

As used herein, the terms “drug”, “API”, “pharmaceutical drug” and “pharmaceuticals” are used interchangeably and refer to a composition comprising a chemical or biological compound which has a physiological effect when administrated in a subject in need (e.g., the active pharmaceutical ingredient or API), and a pharmaceutical acceptable carrier. In certain embodiments, the term “drug” refers to an API. In other embodiments, in which aversion is caused by a component of the pharmaceutical composition other than the API, the term “drug” encompasses any ingredient of the composition, including but not limited to the API. As used herein, a physiological effect refers to stopping or reversing progression of a disease (e.g., infection with bacteria, plasmodia, and helminths). The physiological effect might include but not limited to clearing a parasite, a bacterium, a fungal, or a virus, from the subject.

As used herein, the term “API” is short for active pharmaceutical ingredient and refers to a chemical or biological compound which has a physiological effect when administrated in a subject in need. In one embodiment, the API is selected from the group consisting of anti-malarial, anti-protozoal, anti-parasitic, anti-viral, anti-retroviral, anti-bacterial, and anti-fungal, anti-cold and flu symptoms, anti-algesia, and anti-allergy drugs. In one embodiment, the API is in its levorotatory form. In another embodiment, the API is in its dextrorotatory form. In yet another embodiment, the API is a racemic mixture of levorotatory form and dextrorotatory form.

In one embodiment, the API is a major pharmaceutical compound for treating algesia; worms, viral or bacterial infections; or, cold, flu or allergy symptoms. In one embodiment, the API is selected from the group consisting of Praziquantel, Piperaquine, Dihydroartemisinin, Ritonavir, Tenofovir, Acetaminophen, Diphenhydramine, Nicotine, Caffeine, Dextromethorphan, Guaifenesin and Loratidine. In one embodiment, the API is an Over the Counter (OTC) pharmaceutical or a drug sharing both structural and functional similarities thereto.

In one embodiment, the API is an anti-algesia or analgesic compound, for example Paracetamol (acetaminophen) or Nonsteroidal anti-inflammatory drug (NSAIDs). Other suitable anti-algesia compound can be found at e.g., www.drugs.com/drug-class/analgesics.html and en.wikipedia.org/wiki/Analgesic.

In one embodiment, the API is an anti-parasitic compound, for example, Antiprotozoals, Antihelminthic, Antinematodes, Anticestodes, Antitrematodes, Antiamoebics, and Antifungals. Other suitable anti-parasitic compound can be found at e.g., F. Matthew Kuhlmann, James M. Fleckenstein, 157-Antiparasitic Agents, Infectious Diseases (Fourth Edition), 2017, Pages 1345-1372.e2, Volume 2, Available online 12 Aug. 2016; en.wikipedia.org/wiki/Antiparasitic; and www.cyto.purdue.edu/cdroms/cyto2/17/chmrx/anthelmi.htm.

In one embodiment, the API may be an antiviral for example, Abacavir, Acyclovir (Aciclovir). Other suitable anti-parasitic compound can be found at e.g., www.fda.gov/Drugs/DrugSafety/InformationbyDrugClass/ucm100228.htm #ApprovedDrugs, en.wikipedia.org/wiki/List_of_antiviral_drugs, or www.emedexpert.com/lists/antivirals.shtml, or www.drugs.com/drug-class/antiviral-agents.html, www.cdc.gov/flu/professionals/antivirals/links.htm, and www.fda.gov/drugs/drugsafety/informationby drugclass/ucm100228.htm.

In one embodiment, the API is an antibacterial, for example, Vancomycin, Aminoglycosides, Aminoglycosides, Ansamycins, Carbacephem, Cephalosporins, Glycopeptides, Lincosamides, Lipopeptide, Macrolides, Monobactams, Oxazolidinones, Penicillins, Penicillin combinations, Quinolones/Fluoroquinolones, Sulfonamides, Tetracyclines, Drugs against mycobacteria, and others. Other suitable antibacterial can be found in a variety of publicly available publications, such as websites, e.g., www.emedicinehealth.com/antibiotics/article_em.htm, or www.drugs.com/article/antibiotics.html, en.wikipedia.org/wiki/List_of_antibiotics, or www.merckmanuals.com/professional/infectious-diseases/bacteria-and-antibacterial-drugs/overview-of-antibacterial-drugs, or www.emedexpert.com/lists/antibiotics.shtml, or www.emedicinehealth.com/antibiotics/article_em.htm #7_types_of_antibiotics, among others.

In one embodiment, the API is an antifungal medication, including but not limited to, Polyene antifungals, Hamycin, Imidazoles, Triazoles, Thiazoles, Allylamines, Echinocandins. Other suitable antifungal medications can be found in publicly available sources, e.g., www.nhs.uk/conditions/antifungal-medicines/, www.drugs.com/drug-class/antifungals.html, https://www.drugs.com/drug-class/topical-antifungals.html, www.livestrong.com/article/27116-list-antifungals/, www.emedexpert.com/lists/antifungals.shtml, www.merckmanuals.com/professional/infectious-diseases/fungi/antifungal-drugs, en.wikipedia.org/wiki/Antifungal, and/or masshealthdruglist.ehs.state. ma.us/MHDL/pubtheradetail.do?id=28.

In one embodiment, the API is an antimalarial medication, including without limitation, e.g., quinine, Chloroquine and chloroquine phosphate, Amodiaquine and its combination with artesunate or sulfadoxine-pyrimethamine; Pyrimethamine and its combination with sulfadoxine. Other suitable antimalarial medication can be found in publicly available sources such as www.mayoclinic.org/diseases-conditions/malaria/diagnosis-treatment/drc-20351190, www.drugs.com/condition/malaria.html, www.drugs.com/drug-class/antimalarial-combinations.html, en.wikipedia.org/wiki/Antimalarial_medication, www.cdc.gov/malaria/travelers/drugs.html, www.nap.edu/read/11017/chapter/11, www.medindia.net/drugs/medical-condition/malaria.htm, www.canada.ca/en/public-health/services/travel-health/drugs-generic-trade-name-treatment-prevention-malaria.html, and/or emedicine.medscape.com/article/221134-medication.

As used herein, structural and functional similarities refer to two or more APIs which share at least about 80% identity of chemical groups and can achieve same physiological effect with a variability of less than about 10% when administered to a subject in need. Conventional methods of analyzing structure and functions of an API are known to one of skill in the art, including but not limited to mass spectrometry (MS), electron microscopy, and various pharmacokinetics and physiological analysis.

In one specific embodiment shown in the examples below, the API is Piperaquine or a drug sharing both structural and functional similarities thereto. In one embodiment, also shown in the examples below, the API is Tenofovir (TAF) or a drug sharing both structural and functional similarities thereto. Other suitable APIs might be selected from among known aversive-tasting APIs.

Bitter taste receptors (TAS2Rs or T2Rs) belong to the superfamily of seven-transmembrane G protein-coupled receptors, which are the targets of >50% of drugs currently on the market. In humans, 25 different bitter taste receptors (T2Rs) are known to be expressed, including TAS2R1, TAS2R3, TAS2R4, TAS2R5, TAS2R7, TAS2R8, TAS2R9, TAS2R10, TAS2R13, TAS2R14, TAS2R16, TAS2R19, TAS2R20, TAS2R30, TAS2R31, TAS2R38, TAS2R39, TAS2R40, TAS2R41, TAS2R42, TAS2R43, TAS2R45, TAS2R46, TAS2R50, and TAS2R60. Known inhibitors of some of these receptors include probenecid (“New Bitter Blocker Discovered.” Science Daily, Jun. 3, 2011 at the website ScienceDaily.com/releases/2011/06/110602122351.htm), certain salts and sweeteners (Slack, J P et al, Modulation of Bitter Taste Perception by a Small Molecule hTAS2R Antagonist, Curr. Biol, 20:1104-1109 Jun. 22, 2010). See also International Patent Appln No. 202019/161/322, incorporated by reference herein; U.S. Pat. No. 5,580,545; and Roland et al, cited supra, among other reports.

As used herein, the term “subject” as used herein means a mammalian animal, including a human, a veterinary or farm animal, a domestic animal or pet. In one embodiment, the subject of these methods and compositions is a human. In a further embodiment, the subject of these methods and compositions is a child. As used herein, “child” or “children” refers to a human whose age is 0 month to 18 years, including a baby who is 0 to 12 months old; a toddler who is 1 to 3 years old; a preschool child who is 3 to 5 years old; a grade-schooler who is 5 to 12 years old; and a teen who is 12 to 18 years old. In certain embodiments, the subject of these methods and composition is an adult. In yet a further embodiment, the subject of these methods and compositions is a senior adult who is beyond 65 years old. Still other suitable subjects include, without limitation, non-murine, rat, canine, feline, porcine, bovine, ovine, non-human primate and others.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language.

As used herein, the term “about” or “~” means a variability of 10% from the reference given, unless otherwise specified.

### 2. Compositions

In one aspect, a composition comprises a compound of Formula A,

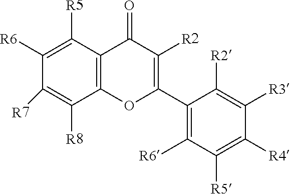

wherein R2, R5, R6, R7, R8, R2′, R3′, R4′, R5′, and R6′, are independently selected from H, OH, a C₁ to C₁₀ alkyl, C₂ to C₁₀ alkenyl, C₂ to C₁₀ alkynyl; C₁ to C₁₀ alkoxy, or a substituted alkyl, alkenyl or alkynyl, or a halogen; and a carrier or excipient, which is pharmaceutically acceptable or safe for consumption or contact by a mammalian subject, e.g., human. This composition suppresses the aversive taste of an ingestible material. In one embodiment, the ingestible material is an active pharmaceutical ingredient (API) which causes aversiveness in a subject upon oral administration. In another embodiment, the ingestible material is a foodstuff, a beverage, an over-the-counter medicine, a prescription medicine, a vitamin and any product or composition placed into the oral cavity for contact or ingestion which would benefit from a reduction in aversive taste. Examples of compounds of Formula A include those in which the substituents are defined as follows: a compound wherein R6 is methyl and R2, R5, R7, R8, R2′, R3′, R4′, R5′, and R6′ are each hydrogen; a compound wherein R5 is OH, R3′ is OH, R4′ is OH and R2, R7, R8, R2′, R5′, and R6′ are each hydrogen; a compound wherein R5 is OCH₃, and R2, R7, R8, R2′, R3′, R4′, R5′, and R6′ are each hydrogen; a compound wherein R6 is OCH₃, R4′ is OH, and R2, R5, R7, R8, R2′, R3′, R5′, and R6′ are each hydrogen. Additional compounds include those wherein R5 is OCH₃, R7 is OCH₃ and R4′ is OCH₃ and R2, R8, R2′, R3′, R5′, and R6′ are each hydrogen and wherein both R5 and R4′ are each OCH₃ and the remaining R groups are hydrogen. In one embodiment, the compound is 6-methylflavone. In other embodiments, the composition contains other blockers of aversiveness which are other flavones, such as 5′3′4′ trihydroxyflavone, 5 methoxyflavone, 4′-hydroxyl-6-methoxyflavone, 4′5 dimethoxyflavone, or 5, 7, 4′ trimethoxyflavone. In another embodiment, the composition contains other blockers of aversiveness which are not flavones.

In one embodiment, the compounds of Formula A inhibit the activity of a TAS2R receptor. In another embodiment, the compounds of Formula A inhibit the activity of two or more TAS2R receptors. In one embodiment the receptor that is blocked or inhibited by the compound of Formula A is TAS2R14. In one embodiment the receptor that is blocked or inhibited by the compound of Formula A is TAS2R39. Still others of the TAS2R family may be blocked by the compounds of Formula A or by a cocktail of blockers including at least one compound of Formula A.

These compositions can be in any desired physical form, depending on their intended use. In one embodiment, the composition is in a liquid form. In another embodiment, the form of the composition may be solid, powder, semi-solid, gel, emulsion or solution. In still other compositions, the compositions are a liquid formulation or pre-rinse designed to be presented orally to the subject prior to ingestion of the ingestible material causing aversiveness, e.g., an API.

In one aspect, the composition in one embodiment contains a compound of Formula A, as described herein, in a suitable pharmaceutical carrier or excipient or formulation. In still another embodiment, the composition comprises a single compound of Formula A or a cocktail of one or more of the different compounds of Formula A.

In an embodiment the composition provided herein contains an effective amount of a blocker of the aversive sensation caused by a selected drug or API or other ingestible material. In another embodiment, the composition contains a cocktail of one or more compounds of Formula A with one or more other TAS2R inhibitor or blocking compounds.

In one embodiment the composition provided herein contains an effective amount of a blocker of the aversive sensation caused by a selected drug or API or other ingestible material. In another embodiment, the composition is an admixture of a compound of Formula A and an ingestible material, in a suitable formulation. In one embodiment, the composition is an admixture of a compound of Formula A and the ingestible material, e.g., 6-methylflavone and an API, e.g., TAF. In one embodiment, where the ingestible material is a toothpaste, the composition is an admixture of the toothpaste components and a compound of Formula A. Still other admixtures can be contemplated given the teachings herein.

In another embodiment, a liquid pre-rinse composition is provided that comprises a compound capable of blocking the activity of TAS2R taste receptors or other taste receptors that mediate an aversive response from a mammalian subject upon contact with the oral cavity, and a suitable pharmaceutically acceptable carrier or excipient. The composition suppresses the aversive taste of an ingestible composition or compound in a suitable ingestible carrier and with optional excipient components comprising one or more of sodium salts, zinc salts, sophorolipids, components derived from milk, milk, and water. The ingestible composition in this composition can be an active pharmaceutical ingredient (API) which causes aversiveness in a subject upon oral administration, a foodstuff, a beverage, an oral care health product, an over-the-counter medicine, a prescription medicine, a vitamin product and any product or composition placed into the oral cavity for ingestion.

For example, in one embodiment, a liquid pre-rinse composition comprises:
(a) one or more compounds of Formula A

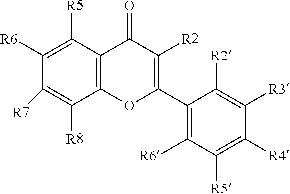

wherein R2, R5, R6, R7, R8, R2′, R3′, R4′, R5′, and R6′, are independently selected from H, OH, a C₁ to C₁₀ alkyl, C₂ to C₁₀ alkenyl, C₂ to C₁₀ alkynyl; C₁ to C₁₀ alkoxy, or a substituted alkyl, alkenyl or alkynyl, or a halogen;
(b) carrier and excipient components comprising one or more of sodium salts, zinc salts, sophorolipids, components derived from milk, milk, and water; and
(c) optional TASR2 blockers other than (a).

In another embodiment, this pre-rinse composition further comprises (d) an API. In one embodiment, such a pre-rinse composition contains the API Tenofovir Alafenamide Fumarate (TAF), and the compound of Formula A 6-methylflavone. This composition suppresses the bitter taste of TAF.

In another embodiment, the compound of Formula A is present at a concentration of about 0.1 to 1 mM 6-methylflavone in a carrier or excipient in a pre-rinse for treatment of a subject prior to contact with the API. In another embodiment, a liquid pre-rinse contains the Formula A blocker, 6-methylflavone in a milk or water formulation. In one embodiment, the formulation also contains a zinc or sodium salt, as described above or a mixture thereof. In another embodiment, the composition contains a sophorolipid. In still another embodiment, the formulation contains components derived from milk, or a combination of these ingredients.

In one embodiment, the composition is a liquid formulation or pre-rinse containing a concentration of between 0.1 micromolar and 1 millimolar of the Formula A compound and/or other aversiveness blockers. In another embodiment, the composition is a liquid formulation or pre-rinse that also contains an effective amount of an API or a drug sharing both structural and functional similarities thereto. In such embodiments, the concentration of the API is about 0.001 mg/mL to about 50 mg/mL.

In one embodiment, provided herein is a composition comprising a drug or API which causes aversiveness in a subject upon oral administration, and an effective amount of a compound having Formula A as described above, wherein the composition suppresses aversiveness of the drug. In certain embodiments, the drug is in a liquid formulation. In other embodiments, the compound is added during preparation of the drug, which may be in a solid or semi-solid or other formulation. Wherever herein the specification refers to a liquid formulation, it is understood that this formulation is an example only and that other formulations are also encompassed. In certain specific embodiments shown in the examples below, the drug/API is Tenofovir Alafenamide Fumarate (TAF; Gilead Sciences). In another embodiment, the API is Piperaquine. In still other embodiments of the compositions, the API component is Praziquantel, Dihydroartemisinin, Zinc Sulfate, or Ritonavir or a drug sharing both structural and functional similarities to one of the above recited API.

One exemplary composition comprises about 0.1 to 1 mM 6-methylflavone in a carrier or excipient in a pre-rinse for treatment of a subject prior to contact with said API. One exemplary composition comprises about 0.1 to 1 mM 6-methylflavone in a carrier or excipient with an effective amount of an API in a pre-rinse for treatment of a subject prior to contact with said API. One such composition is a liquid pre-rinse formulation containing about 1 mM TAF and between 0.1 μM to 1 mM 6-methylflavone, wherein the composition suppresses the bitter taste of TAF. This formulation can be combined with the Na and Zn salts, sophorolipids, milk or water.

Another such composition is a liquid pre-rinse formulation containing about 0.5 mM Piperaquine and between 0.1 μM to 1 mM 6-methylflavone, wherein the composition suppresses the bitter taste of TAF. This formulation can be combined with the Na and Zn salts, sophorolipids, milk or water.

In one embodiment, the API is Praziquantel or a drug sharing both structural and functional similarities thereto, and the blocker compound is 6-methylflavone. In a further embodiment, the effective amount of the block compound is 1 mM in a liquid formulation comprising praziquantel as the API. In some embodiments, Praziquantel or a drug sharing both structural and functional similarities thereto is in its levorotatory form. In some embodiments, Praziquantel or a drug sharing both structural and functional similarities thereto is a racemic mixture.

In certain embodiments, the API is Piperaquine or a drug sharing both structural and functional similarities thereto, and the compound is 6 method flavone. In a further embodiment, the effective amount of 6-methylflavone is 1M in a liquid formulation comprising piperaquine as the API. In yet a further embodiment, the concentration of Piperaquine or a drug sharing both structural and functional similarities thereto is about 4 mg/mL to about 32 mg/mL. In some embodiment, the concentration of Piperaquine or a drug sharing both structural and functional similarities thereto is about 4 mg/mL, about 8 mg/mL, about 16 mg/mL, about 26 mg/mL, or about 32 mg/mL. In certain embodiments, the aversiveness is selected from the group consisting of bitterness, sourness, astringency and nausea. Conventional methods of measuring and quantifying aversiveness is known to one of skill in the art, e.g., Keast, R. S. J. and P. A. S. Breslin. (2002) “Modifying the bitterness of selected oral pharmaceuticals with cation and anion series of compounds.”

Pharmaceutical Research, 19, 1019-1026. PMID: 12180534 Given the teachings herein, one of skill in the art can readily combine other effective amounts of selected API with a blocker compound of Formula A or a cocktail of same.

Any embodiment of the composition provided herein may be utilized in any other embodiment, composition, method or in a kit with other components of the compositions described herein.

### 3. Methods and Kits

In another aspect, a kit for use in a method for suppressing the bitter taste of an active pharmaceutical ingredient (API) comprises two or more of the following components:
(a) one or more compounds of Formula A, as described above;
(b) carrier and excipient components comprising one or more of sodium salts, zinc salts, sophorolipids, components derived from milk, milk, and water;
(c) optional TASR2 blockers other than (a);
(d) an API (or other ingestible material);
(e) instructions for combining (a)-(c) or (a)-(d) to make a pre-rinse;
(f) instructions for combining (a) and (d) or (a) through (d) to make a second composition for oral administration; and
(g) instructions for conducting a drug regimen including administering a pre-rinse prior to administering said second composition to make the taste of the API more palatable to a subject. Optionally, these instructions can optionally contain instructions for admixing the components in a form other than liquid where the ultimate use is to reduce or suppress aversiveness in other than an API.

In one embodiment, a kit is provided herein comprising an effective amount of a compound of Formula A or multiple such compounds as described herein to mix with a drug/API, with Na or Zn salts, and carriers such as sophorolipids, milk or water as described herein to suppress aversiveness of the drug upon oral administration. The kits also can contain the various API for further admixture with the compositions containing compounds of Formula A.

In another embodiment, a method comprises preparing the compositions as described herein (for example from the individual components of a kit) and administering the composition as described below.

One such method for suppressing the taste of an active pharmaceutical ingredient (API) which causes aversiveness in a subject upon oral administration comprises administering to a mammalian subject in need thereof a pre-rinse composition comprising: one or more compounds of Formula A as described above; one or more carrier and excipient components comprising one or more of sodium salts, zinc salts, sophorolipids, components derived from milk, milk, and water; and one or more optional TAS2R blockers other than the compound of Formula A. The method also comprises subsequently administering to said subject the API or a composition comprising the API.

In another embodiment, a method of suppressing the taste of an active pharmaceutical ingredient (API) which causes aversiveness in a subject upon oral administration comprises administering to said subject a pre-rinse composition comprising: one or more compounds of Formula A as described herein; one or more carrier and excipient components comprising one or more of sodium salts, zinc salts, sophorolipids, components derived from milk, milk, and water; one or more optional TAS2R blockers other than the compound of Formula A; in admixture with an effective amount of the API. This method then comprises subsequently administering to said patient a second composition comprising an effective amount of the API. This second compound can comprise a compound of Formula A and the API, or additional components including one or more optional TAS2R blockers and carrier and excipient components comprising one or more of sodium salts, zinc salts, sophorolipids, components derived from milk, milk, and water, or other such pharmaceutically acceptable components depending upon the formulation of the second composition.

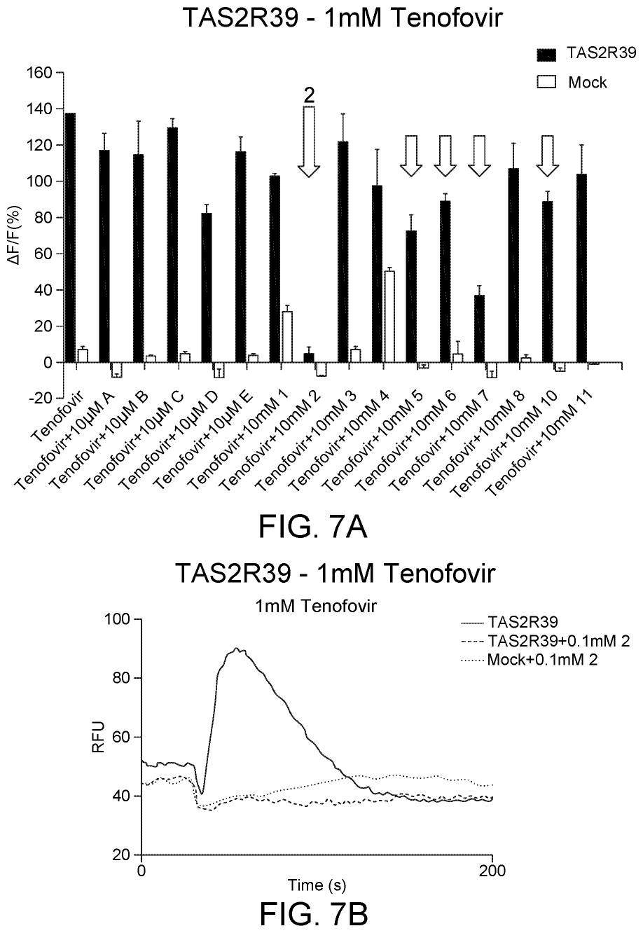

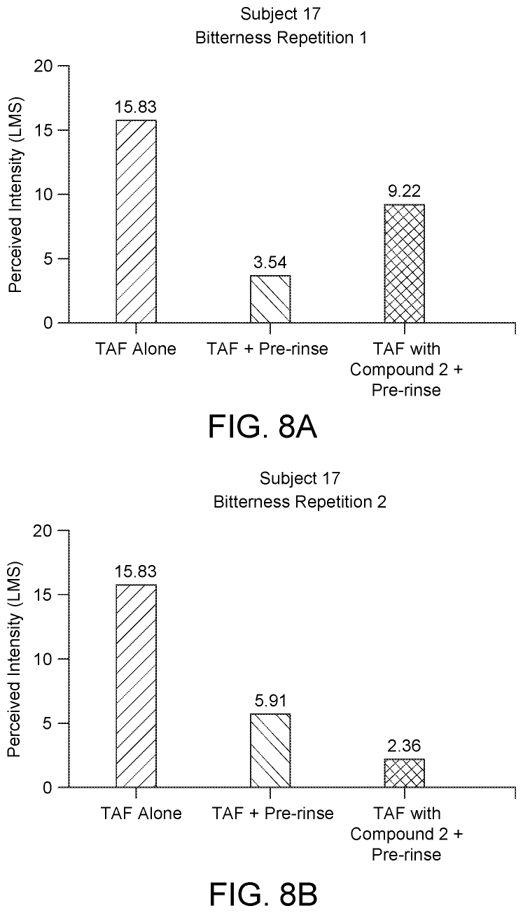

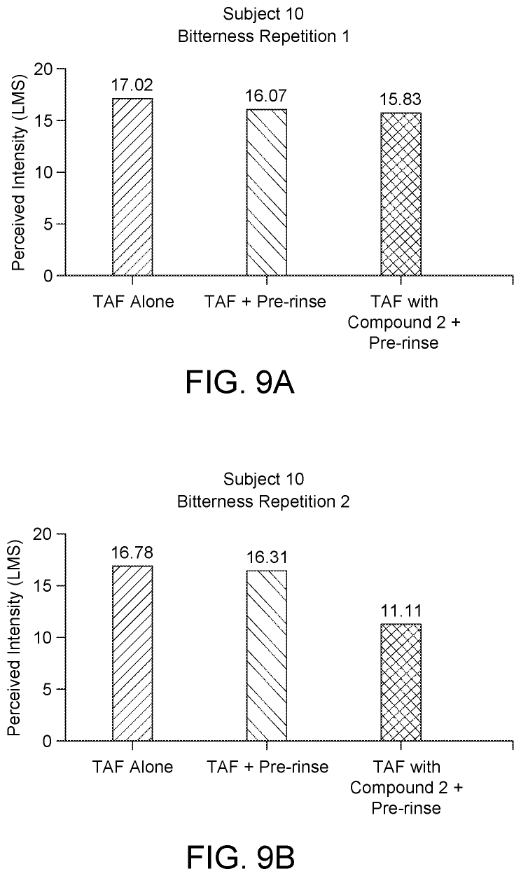

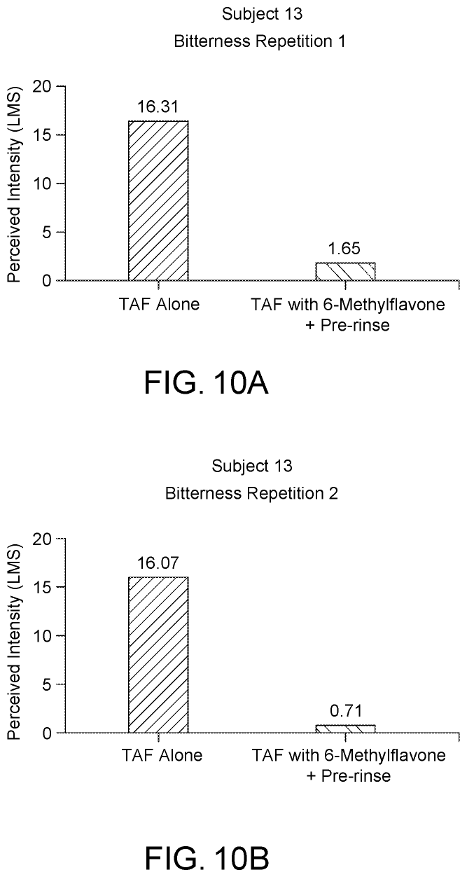

In yet another aspect, a method of suppressing the taste of an active pharmaceutical ingredient (API) which causes aversiveness in a subject upon oral administration comprises a variety of administration regimens. In one embodiment, a liquid formulation of a composition comprising a compound of Formula A (e.g., 6-methylflavone), with a carrier or excipient selected from salts, sophorolipids, milk components or water, is administered as a pre-rinse to a subject prior to oral administration of an API, such as TAF or Piperaquine (See FIGS. 7-10). After the pre-rinse is used to coat the oral cavity, the API is administered. Yet another regimen involves a pre-rinse comprising a cocktail of blocker compounds, salts and carriers administered prior to contact with the selected API.

In another embodiment, the compositions contain both the Formula A blocker compound (or a cocktail of different TAS2R blocker compounds), the salts, and carriers in admixture with an effective amount of the API. This pre-rinse is used to coat the oral cavity and then the selected API is in a second composition alone or in admixture with the same or different compound of Formula A.

The methods of suppressing aversiveness and encouraging consistent ingestion of a prescribed regimen of an API can include administering a pre-rinse composition containing a compound of Formula A, followed by subsequent administration of the API. In another embodiment, the method includes administering a pre-rinse composition containing a compound of Formula A in combination with the API, followed by subsequent administration of a second composition comprising the API.

In still another embodiment, the method includes administering a pre-rinse composition containing a compound of Formula A in combination with the API, followed by administration of a second composition comprising the compound of Formula A in combination with the API. In one such latter method the concentrations of the compound of Formula A in the pre-rinse and in the second composition are the same. In another embodiment, the concentrations of the compound of Formula A in the pre-rinse and in the second composition are different. In still another embodiment of this method the concentrations of the API in the pre-rinse and in the second composition are the same. In still another embodiment of this method, the concentrations of the API in the pre-rinse and in the second composition are different.

Additional embodiments include methods to suppress aversiveness upon oral administration of a drug in a subject. In one embodiment, the compound of Formula A (alone or in a cocktail of blockers) is added to the formulation of the drug/API upon administration. In another embodiment, the compound is administered to the subject prior to the administration of the drug.

Although the methods described immediately above refer to the ingestible material being an API, it is clear from this disclosure that the same components are useful in a method of suppressing the taste of an ingestible material which causes aversiveness in a mammalian subject upon oral administration. Such a method comprises administering to a mammalian subject in need thereof a composition comprising one or more compounds of Formula A as described herein and a suitable carrier and excipients safe for human ingestion. The ingestible material can be any of those disclosed above including oral care products, dental care products, vitamins, foodstuffs or beverages. The compound of Formula A is administered before the ingestible material is ingested by the subject or substantially simultaneously with the ingestion of the ingestible material or is admixed with the ingestible material.

Similarly, the pre-rinse, kit and method embodiments described above useful with a compound of Formula A can be modified to include any bitter blocker that is more suitable than a compound of Formula A for suppressing the bitter or aversive qualities of an ingestible material.

Any embodiment of the method or the kit provided herein may be utilized in any other embodiment, composition, method or kit described herein given the teachings contained in this specification.

The following examples are provided to illustrate certain aspects of the claimed invention. The invention is not limited to these examples.

### Example 1—Sensory Profiles of Target Medications and Validation of Taste Blocking Compounds

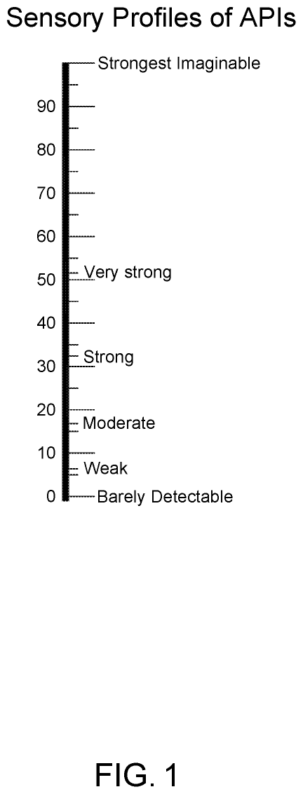

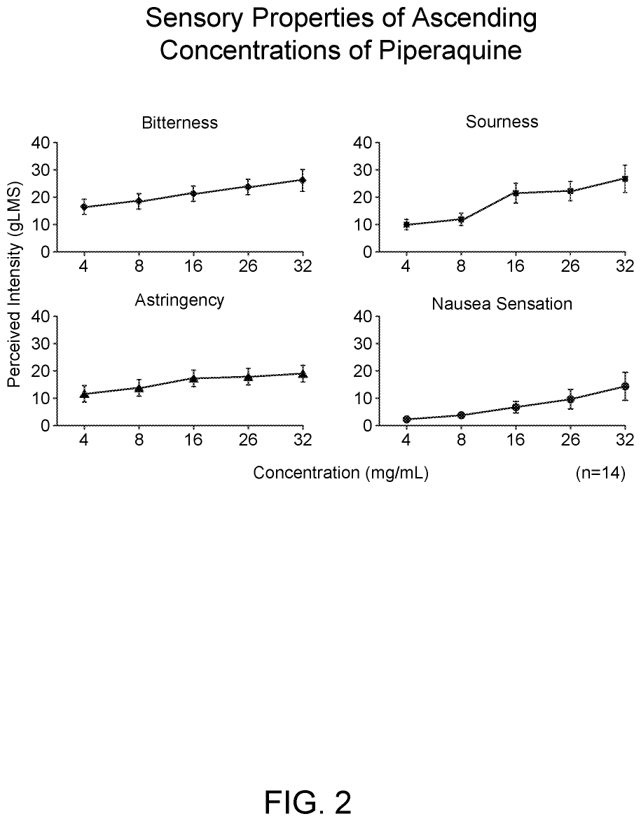

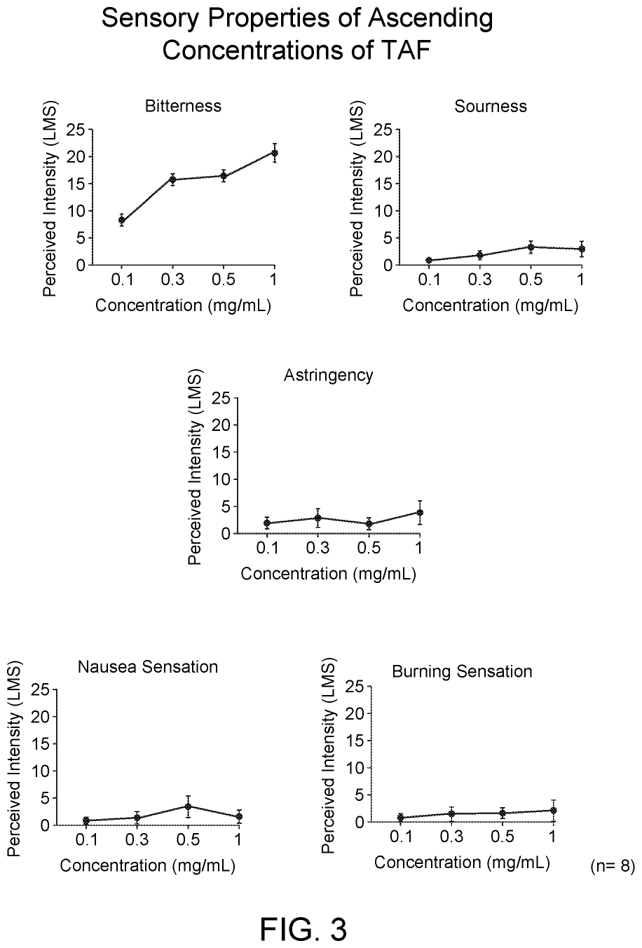

Sensory profiles were generated for all target APIs. See, FIGS. 1-3. As we expected, bitterness is only one source of the noxious sensory properties of medications. Piperaquine was described as having an initial sour and astringent sensation followed by a strong bitter aftertaste. Racemic praziquantel was not perceived as bitter as piperaquine but was stronger than dihydroartemisinin. L-praziquantel was slightly less bitter than the racemic form. Zinc sulfate was predominantly astringent. Both HIV (TAF and ritonavir) medications were strongly bitter. Testing is repeated at lower dosages and with longer tasting intervals to confirm results. Some APIs were bitter and nauseating. As we had previously observed with other stimuli, perceived bitterness and nausea were not correlated.

Piperaquine was both bitter tasting and elicited nausea. Dihydroartemisinin was very bitter and not nauseating. Praziquantel was bitter and not nauseating. Zinc sulfate was mildly bitter and was more nauseating than bitter. TAF and Ritonavir were strongly bitter but not strongly nauseating.

Therefore, the mechanism of eliciting nausea from oral stimulation with these stimuli is separate from bitterness across stimuli. It is possible that for some compounds bitterness and nausea share a common mechanism and for other compounds only bitterness or nausea is operating.

### Example 2

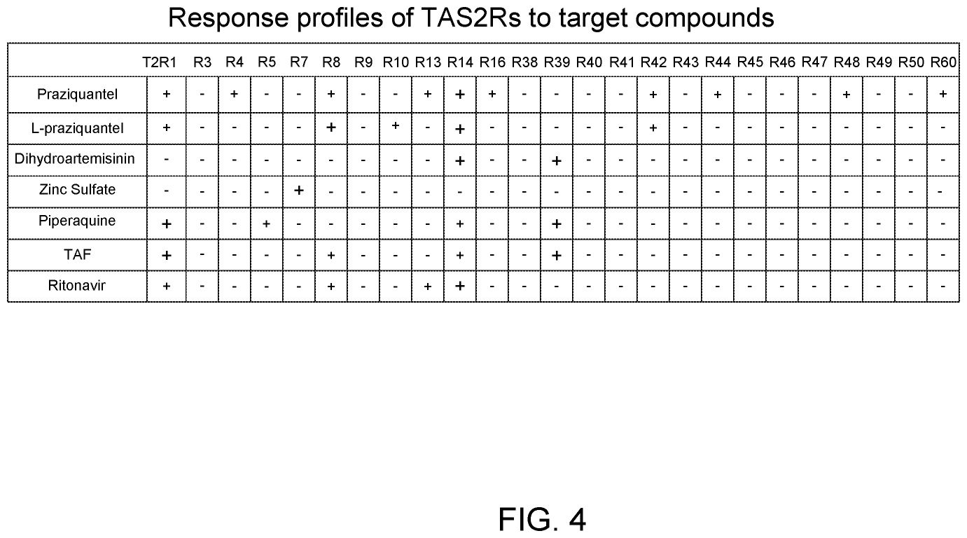

Using a primary hTBEC platform, a high throughput screening (HTS) was conducted with more than 2,000 small molecular weight purified phytochemicals and botanical extracts. The screen identified 40 antagonists that suppressed taste cell activation by target APIs. Only a few receptors showed strong responses to the APIs. All drugs except zinc activate TAS2R14 receptor, which is one of most responsive receptors for praziquantel (both racemic and L-form), dihydroartemisinin and ritonavir. TAS2R39 is one of the most responsive receptors for piperaquine, tenofovir, and dihydroartemisinin. TAS2R1 is responsive to piperaquine and tenofovir. (See FIG. 4)

Therefore, we selected these receptors for validation experiments. It may be that only several bitter taste receptors are responsible for most of the bitter taste of these APIs and possibly other pharmaceuticals.

These compounds were used to validate bitter taste antagonists of the human Taste Bud Epithelial Cells (hTBEC) bioassay responses stimulated by tenofovir alafenamide fumarate (TAF), racemic praziquantel, and piperaquine and yielded a panel of bitter taste antagonists of which 6-methylflavone was quite potent and effective at blocking the stimulation of hTBEC cells by bitter tasting drugs. After 6-methylflavone progressed through a primary assay and several validation bioassays, it was determined that 6-methylflavone was a powerful antagonist of TAS2R39 stimulated by either piperaquine or TAF.

### Example 3

Methods of evaluating the sensory properties of the API and admixtures with the blocker compounds of Formula A are adopted from Breslin PA, and Tharp CD, “Reduction of saltiness and bitterness after a chlorhexidine rinse,” Chem Senses. 2001 February; 26(2):105-16. More details are illustrated below.

Subjects participate in the study after providing informed consent. Gender of the subjects is balanced between male and female. The subjects are adults with ages ranging from 20 years old to 50 years old. The only exclusion criteria, since it is a rinse and spit study, is the ability to follow instructions, demonstrate proper use of the labeled magnitude scale, and to taste. The participants are asked to refrain from eating, drinking, or chewing gum for 1 hr prior to testing. Subjects are trained to use the General Labeled Magnitude Scale (gLMS) following standard published procedures. See. e.g., Breslin P A, and Tharp C D. “Reduction of saltiness and bitterness after a chlorhexidine rinse,” Chem Senses. 2001 February; 26(2): 105-16; Keast and Breslin, “Bitterness suppression with zinc sulfate and Na-cyclamate: a model of combined peripheral and central neural approaches to flavor modification.” Pharm Res. 2005 November; 22(11):1970-7. Epub 2005 Aug. 26; Green et al, “Evaluating the ‘Labeled Magnitude Scale’ for measuring sensations of taste and smell.” Chem Senses. 1996 June; 21(3):323-34; Barry G. Green, et al, “Derivation and evaluation of a semantic scale of oral sensation magnitude with apparent ratio properties,” Chemical Senses, Volume 18, Issue 6, 1 Dec. 1993, Pages 683-702; and Bartoshuk L M, “Comparing sensory experiences across individuals: recent psychophysical advances illuminate genetic variation in taste perception.” Chem Senses. 2000 August; 25(4):447-60.

The selected API (in racemic or L form as applicable) is diluted/dissolved in deionized (di) filtered water upon use into various concentrations. API at selected concentrations are also prepared in 1 mM of the selected Compound of Formula A, e.g., 6-methylflavone. The API is prepared and dissolved in deionized filtered water or in 1M 6-methylflavone to a final concentration. All solutions are stored on ice or at 4° C. to 8° C. and brought up to room temperature prior to testing.

The following testing protocol is performed. The tested solutions are provided to the subjects in random order. Subjects rinsed with di water at least four times over a 2-minute period prior to tasting each solution. The subjects are instructed to wear nose clips to eliminate olfactory input, pour the whole solution in their mouth for 3-5 seconds, and rate the perceived sensation (including bitterness, sourness, astringency, burning, stinging and nausea) intensity of the solution while it remained in the mouth, prior to expectorating. Taste intensity is recorded on a computerized gLMS, analyzed and plotted.

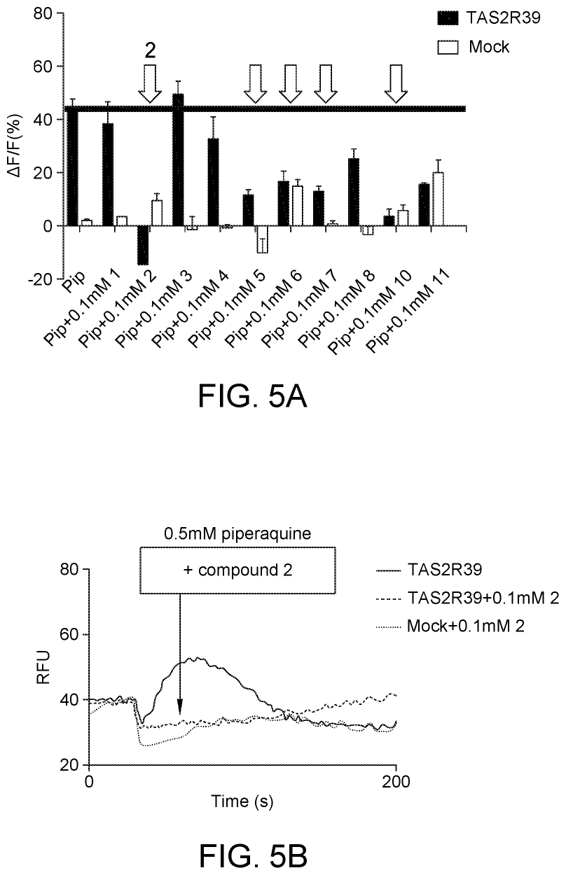

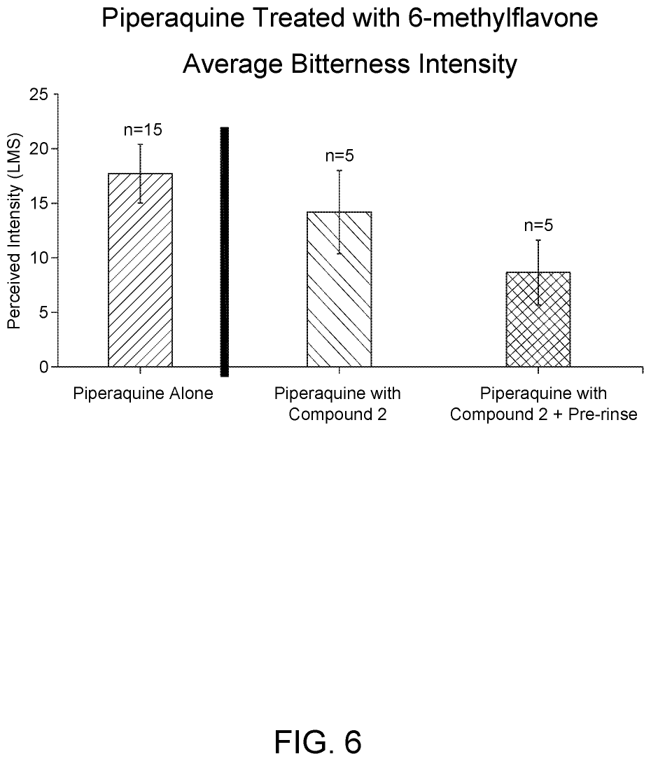

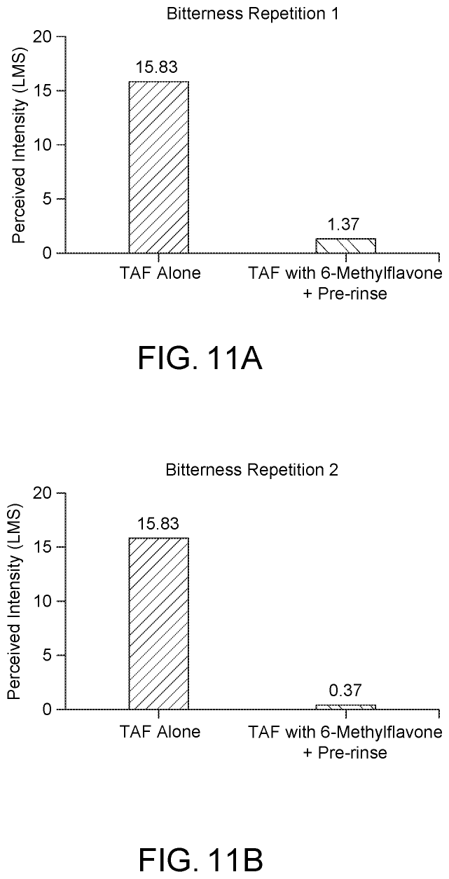

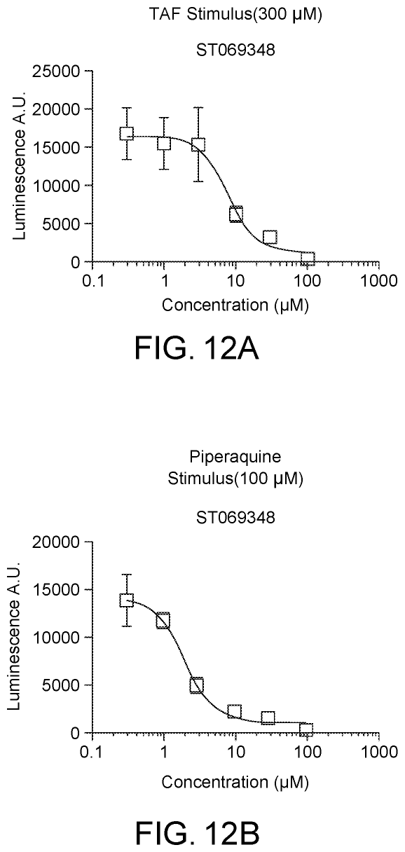

See the results in FIGS. 5A to 11B.

In parallel, 6-methylflavone was found to be an effective antagonist in a subset of human subjects in blocking the bitter taste of piperaquine and TAF in an admixture. The blocking effect was stronger for both TAF and piperaquine when a pre-rinse with the blocker was added. In summary, this ‘trifecta’ of positive results in the hands of 3 different laboratories showed that 6-methylflavone was an effective antagonist for both TAF and piperaquine. It is further anticipated that this Formula A blocker compound may block all agonists of TAS2R39.

We also successfully demonstrated that sodium, zinc or milk added as sensory excipients to APIs also significantly reduce their bitterness.

Dose-response testing of the blocker with the two drugs/API is performed to determine the optimum concentration for maximal blocking within and across individual subjects. The temporal parameters of a blocker pre-rinse that maximizes blocking within and across individual subjects is studied. Other excipients are added in a cocktail with the blocker to further reduce the overall bitterness of the two drugs.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A method of suppressing the taste of an active pharmaceutical ingredient (API) which causes aversiveness in a subject upon oral administration comprises:
(a) orally administering to the subject in need thereof a pre-rinse composition comprising:
i. one or more compounds of Formula A 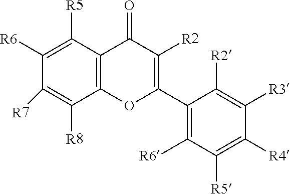
wherein R2, R5, R6, R7, R8, R2′, R3′, R4′, R5′, and R6′, are independently selected from H, OH, a C₁ to C₁₀ alkyl, C₂ to C₁₀ alkenyl, C₂ to C₁₀ alkynyl; C₁ to C₁₀ alkoxy, or a substituted alkyl, alkenyl or alkynyl, or a halogen;
ii. carrier and excipient components comprising one or more of sodium salts, zinc salts, sophorolipids, milk or a milk product, and water; and
iii. one or more optional TAS2R blockers other than (i), and
(b) orally administering to the subject the API or a composition comprising the API; wherein the API or a composition comprising the API is administered subsequently following orally administering the pre-rinse composition, wherein the compound of Formula A is 6-methylflavone.

2. The method of claim 1, wherein the API or the composition comprising the API is a foodstuff, a beverage, an over-the-counter medicine, a prescription medicine, a vitamin and any product or composition placed into the oral cavity for ingestion.

3. The method of claim 1, wherein the API is selected from the group consisting of anti-malarial, anti-protozoal, anti-parasitic, anti-viral, anti-retroviral, anti-bacterial, anti-fungal, anti-cold and flu symptoms, anti-analgesia, and anti-allergy drugs.

4. The method of claim 1, wherein the API is Tenofovir Alafenamide Fumarate (TAF), Praziquantel, Piperaquine, Dihydroartemisinin, Zinc Sulfate, or Ritonavir or a drug sharing both structural and functional similarities to one of said API.
